(19) United States
(12) Patent Application Publication
(10) Pub. No.: US 2024/0000320 A1
(43) Pub. Date: Jan. 4, 2024

(54) MEDICAL INSTRUMENT FOR USE IN A TELEMEDICINE APPLICATION TO SENSE HEART RATE, LUNG, ABDOMINAL SOUNDS, BLOOD PRESSURE, PULSE, OXYGEN SATURATION, AND RESPIRATORY RATE EVALUATION

(71) Applicant: Kevin S. Merigian, Eads, TN (US)

(72) Inventor: Kevin S. Merigian, Eads, TN (US)

(21) Appl. No.: 17/854,275

(22) Filed: Jun. 30, 2022

(51) Int. Cl. A61B 5/0205; A61B 5/00

(52) U.S. Cl. CPC A61B 5/0205; A61B 5/0022

(57) ABSTRACT

A medical instrument for use in telemedicine application including an elongated member connected to a central member. The central member includes a diaphragm at one end and a bell at another end, each used for different listening sounds of a living subject such as an animal or human body. The medical instrument amplifies audio signals up to forty-five times associated with a patient's heartbeat, breathing, blood flow, digestion, or any other biological process that produces audio signals. The medical instrument records electrical heart tracings (ECG tracings) and super-imposes the digital visual representation of the audio signals from heart simultaneously. The medical instrument transmits data remotely to a server and/or a user device for analysing the heart rate, rhythm (both regular and irregular) and changes that could suggest atrial fibrillation, tachyarrhythmia, dissociations, ischemia and ventricular contractions. The medical instrument acts as an ear free listening device and a heart rate monitor.

### FIELD OF THE INVENTION

The present invention generally relates to a medical instrument. More specifically, the present invention relates to a medical instrument such as an electronic stethoscope or digital stethoscope for use in a telemedicine application, e.g., for monitoring heart rate, heart, lung, abdominal sounds, bruits, blood pressure, pulse, oxygen saturation, and respiratory rate evaluation.

### BACKGROUND OF THE INVENTION

It is known that a stethoscope is widely used as a medical device for auscultation, or for listening of the internal sounds of a living subject such as an animal or human body. Generally, the stethoscope is used by a healthcare professional to listen to heart, artery, vein, intestine, lung, and/or bowel sounds. Based on the sounds heard through the stethoscope, the healthcare professional diagnose various illnesses or conditions of the living subject. In addition to the stethoscope, other devices have been developed to detect the sounds of the living subject. Examples of such devices include, but not limited to, electronic devices such as microphones, and transducers.

Several devices for listening of the internal sounds of the living subject have been disclosed in the past. One such example is disclosed in a U.S. Pat. No. 9,973,847, entitled “Mobile Device-Based Stethoscope System” (“the '847 patent”). The '847 patent discloses a mobile device-based stethoscope system that transmits, records, and analyses sounds to generate a list of matching conditions and facilitates easy attachment across various electronic medical record platforms and other means of communication. The invention is configured to allow the use of either an integrated wireless stethoscope, or an in-line adapter for a conventional stethoscope. Patient sounds are sent from the selected stethoscope head to the mobile device having a software application that allows for the analysis, attachment, and further manipulation of the data.

Another example is disclosed in a U.S. Pat. No. 8,827,920, entitled “Telemedical Stethoscope” (“the '920 patent”). The '920 patent discloses telemedical stethoscope, which automatically diagnoses a disease, and records visually and auditorily, and stores the stethoscope data on a screen. The '920 patent enhances primary diagnosis and treatment effect for a patient by transmitting/receiving the data to/from a doctor at a medical center and by receiving a telemedicine service. In addition, the '920 patent transmits the data to a health management program so as to be used for personal healthcare and disease prognosis decisions of a patient.

Another example is disclosed in a United States Publication No. 20160066797, entitled “Compound Medical Device” (“the '797 Publication”). The '797 Publication discloses a compound medical device, which accurately estimates the disease state of a subject or determines the gestational week or health status of a subject, may include an auscultation unit, receiving bodily sound, a body temperature measurement unit, measuring the body temperature of a subject, a controller unit, connected to the auscultation unit and the body temperature measurement unit and estimating the disease state or determining the gestational week or health status of the subject based on the input received from the auscultation unit and the body temperature measurement unit, and an output unit, outputting the disease state or the gestational week or health status of the subject estimated or determined by the controller unit.

Although the above disclosures provide diagnosis of a disease, record, store and transmit the data from the stethoscope, they have few problems. For example, the current healthcare system has been slow to embrace patient-centered care. Hospitals and clinics have created sterile and inhospitable atmospheres for patient diagnosis and care. Further, it takes considerable time to evaluate the patient/living subject. Furthermore, some of the known devices cannot be used by the healthcare professionals due to the conditions at the hospitals and clinics since the Coronavirus (Covid-19) pandemic. Further, the existing devices cannot acquire electrocardiogram (ECG) tracings. Therefore, electrocardiographic data acquisition or heart tracings cannot be measured nor transposed simultaneously on a display with sound data or other data.

Therefore, there is a need for a medical instrument that will allow the healthcare professionals to assess and integrate the vital signs, heart, lungs, abdominal sounds, blood pressure and pulse in patients at their homes or work office.

### BRIEF SUMMARY OF THE INVENTION

It is an object of the present invention to provide a medical instrument for use in a telemedicine application that avoids the drawback of known stethoscopes.

It is another object of the present invention to provide a medical instrument that allows the healthcare professionals to assess the vital signs, heart, lungs, breath sounds, bruits, abdominal sounds, blood pressure, pulse, eyes, and skin in patients at their homes or work office.

It is another object of the present invention to provide a medical instrument that acts as an ear free listening device and a heart rate monitor.

In order to overcome the limitations here stated, the present invention provides a medical instrument for use in a telemedicine application. The medical instrument includes an elongated member connected to a central member. The central member includes a diaphragm at one end and a bell at another end, each used for different listening sounds of a living subject such as an animal or human body. The medical instrument amplifies audio signals up to forty-five times associated with a patient's heartbeat, breathing, blood flow, digestion, or any other biological process that produces audio signals. Further, the medical instrument records electrical heart tracings (ECG tracings).

The medical instrument super-imposes the digital visual representation of the audio signals from heart and the ECG tracings. The medical instrument transmits data remotely to a server and/or a user device for analysing the heart rate, rhythm (both regular and irregular) and changes that could suggest atrial fibrillation, tachyarrhythmia, dissociations, ischemia and ventricular contractions.

In one advantageous feature of the present invention, the medical instrument acts as a portable ear free listening device and a heart rate monitor.

In another advantageous feature of the present invention, the medical instrument is compatible with third party telehealth service providers for home use and allows patients to communicate with healthcare professionals in real-time. The medical instrument connects to the server and helps to analyse the heart rate, rhythm (both regular and irregular) and changes, and connects the patient to current therapies for heart diseases and encourages lifestyle changes as well as supplements and medications for healthcare management.

In another advantageous feature of the present invention, the medical instrument allows the healthcare professionals to assess the vital signs, heart, lungs, abdominal sounds, blood pressure and pulse in patients at their homes or work office.

In yet another advantageous feature of the present invention, the medical instrument displays S1, S2, S3, S4, murmurs, heaves thrills, arterial bruits, systolic and diastolic blood pressure changes, lung sounds, tracheal sounds (stridor), breath sounds in the peripheral lung fields (crackles, rhonchi, wheezing), oxygen saturation and abdominal sounds (borborygmi). In addition, the medical instrument allows the user or operator to transpose heart sounds, breath sounds, blood pressure measurement with pulse oximetry readings, body temperature, and ECG tracings for displaying at the display screen.

Features and advantages of the invention hereof will become more apparent in light of the following detailed description of selected embodiments, as illustrated in the accompanying FIGUREs. As will be realized, the invention disclosed is capable of modifications in various respects, all without departing from the scope of the invention. Accordingly, the drawings and the description are to be regarded as illustrative in nature.

### BRIEF DESCRIPTION OF THE DRAWINGS

The present invention will now be described in detail with reference to the drawings, which are provided as illustrative examples of the invention as to enable those skilled in the art to practice the invention. It will be noted that throughout the appended drawings, like features are identified by like reference numerals. Notably, the FIGUREs and examples are not meant to limit the scope of the present invention to a single embodiment, but other embodiments are possible by way of interchange of some or all of the described or illustrated elements and, further, wherein:

FIG. 1 illustrates an environment of a medical instrument used by a healthcare professional on a living subject such as an animal or human body for listening to the heart, in accordance with one exemplary embodiment of the present invention;

FIGS. 2A, 2B, 2C, 2D, 2E and 2F show a top view, a right side view, a left side view, a rear view, a front view, and a bottom view, respectively of a medical instrument, in accordance with one embodiment of the present invention;

FIG. 3 shows a block diagram of the medical instrument communicatively connected to a server and a user device, in accordance with one embodiment of the present invention;

FIG. 4 shows an internal design on a medical instrument, in accordance with one exemplary embodiment of the present invention;

FIG. 5 shows a display showing breath sounds and lung sounds, in accordance with one exemplary embodiment of the present invention;

FIG. 6 shows a display showing ECG heart signal and corresponding ECG waveform, in accordance with one exemplary embodiment of the present invention; and

FIG. 7 shows a network architecture of a medical instrument communicating with a server operated/managed by a telemedicine provider/hospital/government/third party telehealth service provider, in accordance with one exemplary embodiment of the present invention.

### DETAILED DESCRIPTION OF THE EMBODIMENTS

The detailed description set forth below in connection with the appended drawings is intended as a description of exemplary embodiments in which the presently disclosed invention may be practiced. The term “exemplary” used throughout this description means “serving as an example, instance, or illustration,” and should not necessarily be construed as preferred or advantageous over other embodiments. The detailed description includes specific details for providing a thorough understanding of the presently disclosed medical instrument. However, it will be apparent to those skilled in the art that the presently disclosed invention may be practiced without these specific details. In some instances, well-known structures and devices are shown in functional or conceptual diagram form in order to avoid obscuring the concepts of the presently disclosed medical instrument.

In the present specification, an embodiment showing a singular component should not be considered limiting. Rather, the invention preferably encompasses other embodiments including a plurality of the same component, and vice-versa, unless explicitly stated otherwise herein. Moreover, the applicant does not intend for any term in the specification to be ascribed an uncommon or special meaning unless explicitly set forth as such. Further, the present invention encompasses present and future known equivalents to the known components referred to herein by way of illustration.

Although the present disclosure provides a description of a medical instrument for use in a telemedicine application, it is to be further understood that numerous changes may arise in the details of the embodiments of the medical instrument. It is contemplated that all such changes and additional embodiments are within the spirit and true scope of this disclosure.

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure.

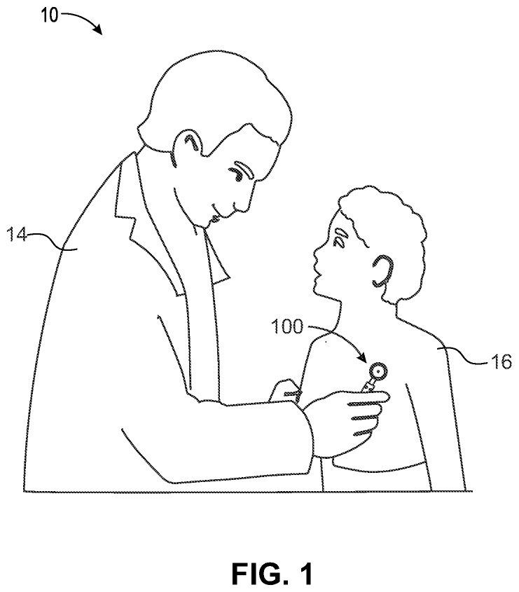

In one embodiment, the present invention discloses a medical instrument for use in a telemedicine application. FIG. 1 shows an environment 10 of a medical instrument 100 used by healthcare professional 14 on a living subject 16 such as an animal or human body for listening to the heart, in accordance with one exemplary embodiment of the present invention. In the present invention, medical instrument 100 operates as an electronic or digital stethoscope for assessing the vital signs, heart, lungs, abdominal sounds, blood pressure and pulse in living subject or patient 16 at their homes or work office. Although it is shown that healthcare professional 14 assesses living subject 16 in FIG. 1, it is obvious to a person skilled in the art that living subject or patient 16 himself/herself or with the help of a caretaker or family member can operate medical instrument 100 without departing from the scope of the present invention.

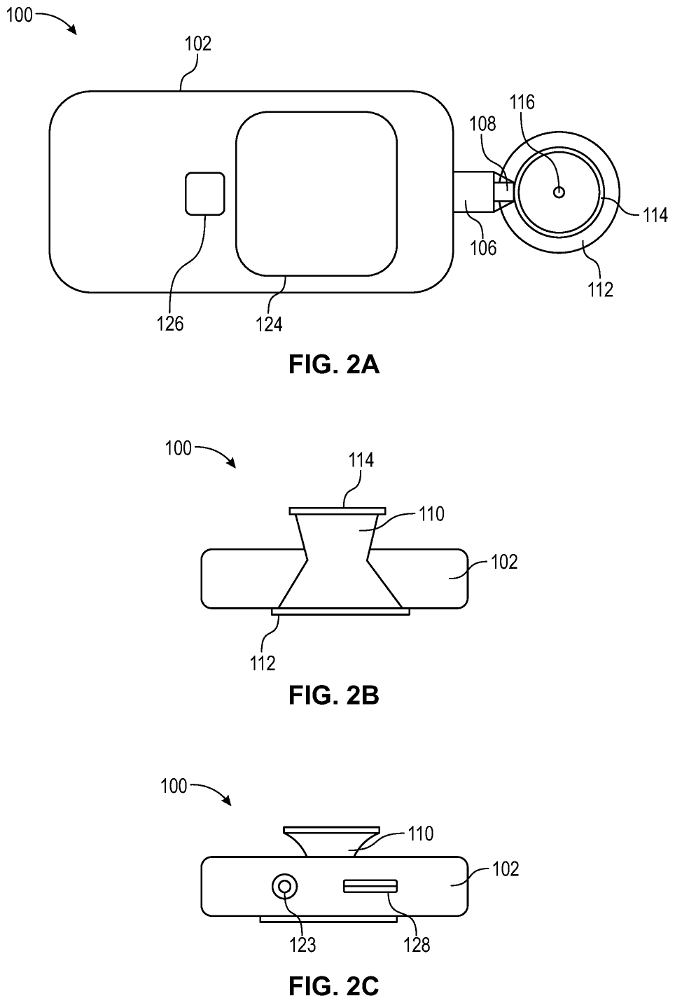

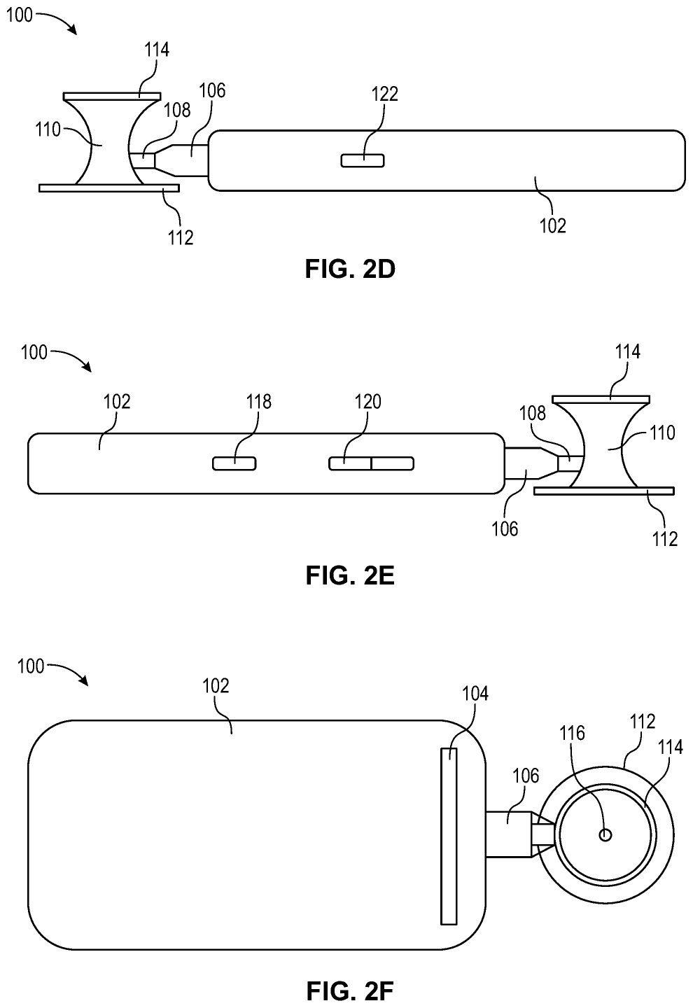

Now referring to FIGS. 2A, 2B, 2C, 2B, 2E and 2F; a top view, a right side view, a left side view, a rear view, a front view and a bottom view, respectively of medical instrument 100 are shown, in accordance with one embodiment of the present invention. Medical instrument 100 includes elongated member 102 made of metal, plastic, wood or any other suitable material. Elongated member 102 comes in a variety of shapes and sizes. In one example, elongated member 102 comes in a rectangular configuration. In another example, elongated member 102 comes in a cylindrical or conical configuration. Elongated member 102 presents a hollow structure and acts as a handle, which healthcare professional 14 or patient 16 uses for operating medical instrument 100. Elongated member 102 encompasses sensor 104 at the bottom as shown in FIG. 2F. Sensor 104 includes, but not limited to, an Electrocardiography (ECG) sensor or ECG strip. Elongated member 102 includes an extending member or stem 106, as shown in at least FIGS. 2A, 2D, 2E and 2F. Extending member 102 is made of metal such as stainless steel, for example. Extending member 102 includes connecting member 108 that helps to connect extending member 102 to central member or drum 110.

Central member 110 presents a substantially hollow concave shaped structure. Central member 110 encompasses diaphragm 112 at one end and bell 114 at another end, as can be seen from at least FIGS. 2B, 2C, 2D and 2E. Diaphragm 112 comes in a relatively flat configuration and bell 114 comes in a concave shape with bell hole or acoustic sound hole 116. FIG. 2A shows bell 114 having bell hole 116 at the center. Further, elongated member 102 encompasses first button 118 and second button 120 at one side, as can be seen from at least FIG. 2E. Further, elongated member 102 encompasses third button 122 at another side. First button 118 indicates a picture or record ON/OFF button for recording sounds of a patient. Second button 120 includes a volume button configured to adjust the sound produced or recorded by medical instrument 100. Third button 122 indicates power ON/OFF button for turning ON/OFF of medical instrument 100. Further, elongated member 102 encompasses first port 123 for connecting an audio jack allowing the user to connect hear/listen the sounds detected by diaphragm 112.

At the top, elongated member 102 encompasses display screen 124 for displaying readings measured by medical instrument 100. Further, elongated member 102 encompasses speaker 126 for announcing the sounds recorded by medical instrument 100. In one implementation, elongated member 102 encompasses second port 128 such as a Universal Serial Bus (USB) port or charging port, as shown in FIG. 2C. Medical instrument 100 operates similar to medical instrument 12, as explained above.

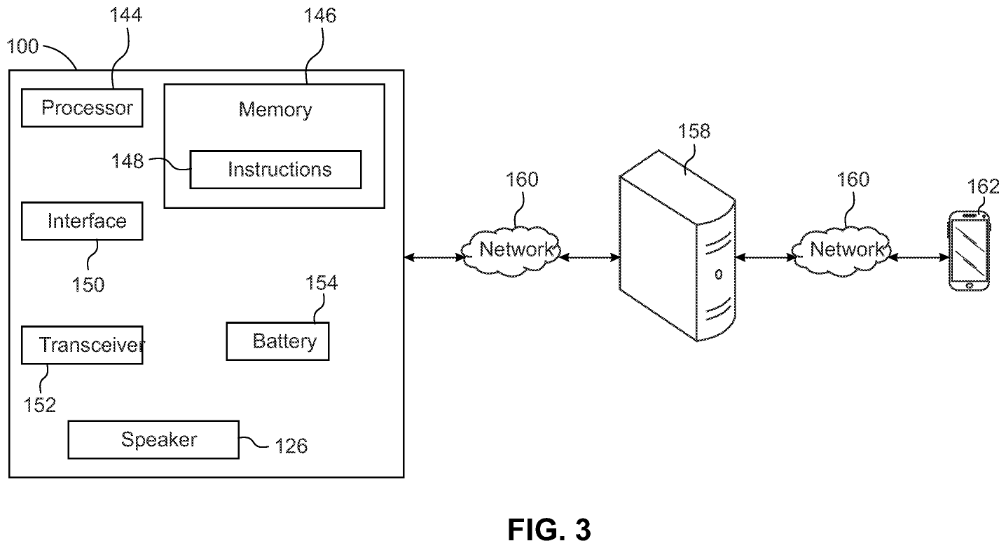

Now referring to FIG. 3, a block diagram of medical instrument 100 is shown, in accordance with one embodiment of the present invention. Medical instrument 100 encompasses processor 144, and memory 146. Processor 144 includes a central processing unit (CPU), a graphics processing unit (GPU) or both. In one example, memory 146 includes instructions 148 stored therein. Instructions 148 may also reside, completely or at least partially, within the memory 146 and/or within the processor 144 during execution thereof. Instructions 148 may further be transmitted or received over a network 160 via transceiver 152 utilizing any one of a number of well-known transfer protocols.

Medical instrument 100 encompasses interface 150 such as hardware and/or software devices/applications used for operating medical instrument 100. Medical instrument 100 encompasses transceiver 152 for sending or receiving instructions/data from other devices such as server 158 or user device 162. Medical instrument 100 further encompasses battery 154 for powering electronic components in medical instrument 100. Second port 128 helps to charge battery 154 of medical instrument 100 or to transfer data from medical instrument 100 to other devices such as server 158 or user device 162.

Medical instrument 100 operates as a standalone device and/or communicatively connects to other devices such as server 158 or user device 162 via network 160. Here, server 158 indicates a central server operated by a hospital, an insurance provider, government, etc. for storing and processing the health data of patient(s) 16. User device 162 indicates a device such as a mobile phone, tablet, laptop, smartwatch, desktop, etc. used by patient 16, healthcare professional 14, or any other individual(s). Network 160 includes a wireless network, a wired network or a combination thereof. Network 160 can be implemented as one of the different types of networks, such as intranet, local area network (LAN), wide area network (WAN), the internet, and the like. The network 160 may either be a dedicated network or a shared network. The shared network represents an association of the different types of networks that use a variety of protocols, for example, Hypertext Transfer Protocol (HTTP), Transmission Control Protocol/Internet Protocol (TCP/IP), Wireless Application Protocol (WAP), and the like, to communicate with one another. Further the network 60 may include a variety of network devices, including routers, bridges, servers, computing devices, storage devices, and the like.

In operation, healthcare professional 14 or patient 16 himself/herself places diaphragm 30 in contact with or in proximity to sample to be examined such as patient's chest, stomach, limb such as an arm or leg, or any other body part of the patient, as shown in FIG. 1, for example. Subsequently, healthcare professional 14 or patient 16 presses third button 122 to power ON medical instrument 100. Here, processor 144 employs diaphragm 112, which amplifies audio signals up to forty-five (45) times associated with a patient's heartbeat, breathing, blood flow, digestion, or any other biological process that produces audio signals. In one implementation, medical instrument 100 toggles between diaphragm 112 and bell 114 for employing different listening techniques. Here, ECG sensor 104 records electrical heart tracings (ECG tracings). In accordance with the present invention, medical instrument 100 super-imposes the digital visual representation of the audio signals from the heart simultaneously with the electrical heart tracings into an audio file. In one example, medical instrument 100 stores the audio file in memory 146. In another example, medical instrument 100 transmits super-imposed heart sound and ECG tracings to server 158 or user device(s) 162 via network 160 through transceiver 152.

In one implementation, medical instrument 100 integrates blood pressure readings with heart sound and ECG tracings using a digital sphygmomanometer (not shown). In accordance with the present invention, medical instrument 100 is capable of recording blood pressure, heart sounds, chest breathing sounds, carotid and other arterial sounds and ECG tracings in the intervals of 15, 30, 45, 60, and 120 intervals depending on the need.

Medical instrument 100 helps to transmit the data in real-time to server 158 and/or user device(s) 162. Healthcare professional 14 and/or insurance provider views the heart sounds, ECG, Blood pressure and Pulse and analyses the heart rate, rhythm (both regular and irregular) and changes that could suggest atrial fibrillation, tachyarrhythmia, dissociations, ischemia and ventricular contractions. Alternatively, medical instrument 100 in conjunction with server 158 analyses the heart rate, rhythm (both regular and irregular) and changes, and connects patient 16 to current therapies for heart diseases and encourages lifestyle changes as well as supplements and medications for healthcare management.

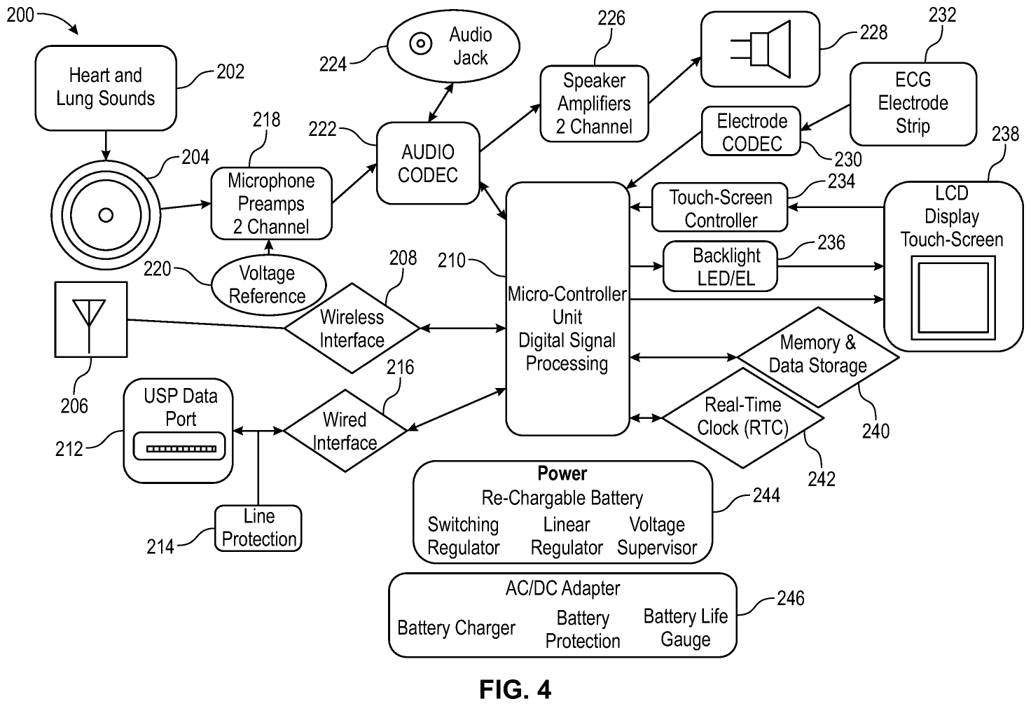

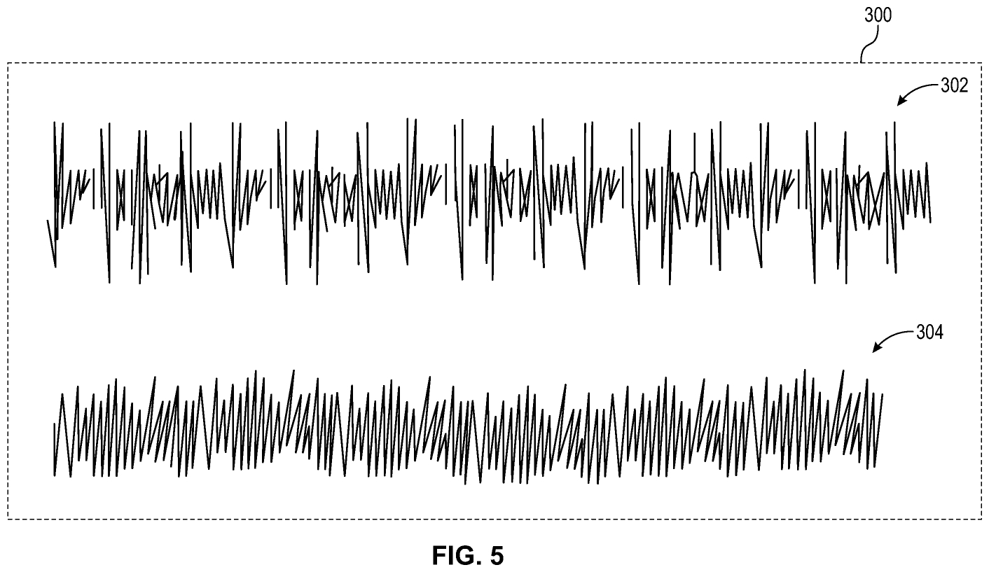

FIG. 4 shows an internal design on medical instrument 200, in accordance with one exemplary embodiment of the present invention. It should be understood that medical instrument 200 implements and operates similar to medical instrument 100, as explained above. Diaphragm 204 configures to hear the sounds produced by heart and lungs (202). Medical instrument 200 includes wireless communication module 206 connected to microcontroller 210 via wireless interface 208. In addition, medical instrument 200 includes first port 212 such as a Universal Serial Bus (USB) port or charging port for charging the electrical components of medical instrument 200 via wired interface 216. In one implementation, medical instrument 200 encompasses line protection circuit 214 for protecting the electrical components from electric fluctuations.

In accordance with the present embodiment, diaphragm 204 detects the sounds produced by heart and lungs and two channel microphone preamp 218 amplifies the sounds using voltage reference 220. Two channel microphone preamp 218 ensures the audio signal is not clipped or distorted and supplies it to audio codec 222 for encoding the audio. Further, audio jack 224 supplies the audio captured from the surroundings.

Microcontroller 210 processes the audio and provides the audio to speaker 228 upon amplifying the audio using two channel speaker amplifiers 226.

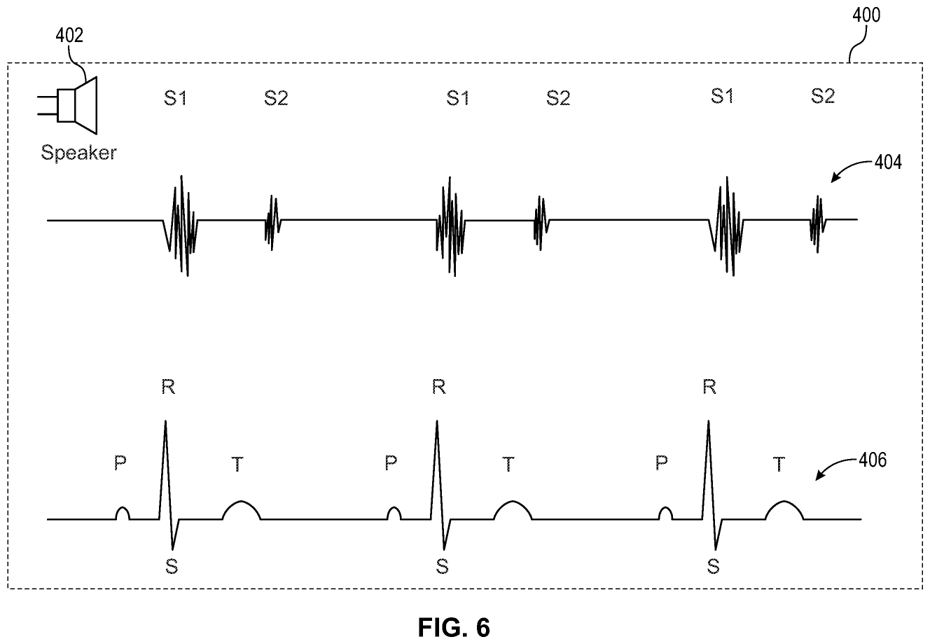

Further, microcontroller 210 receives electrode codec 230 from sensor 232 such as ECG electrode strip positioned at the elongated member (not shown) of medical instrument 200. Furthermore, microcontroller 210 controls and displays information on display screen 238 using touchscreen controller 234 and backlight LED/EL 236. Display screen 238 configures to display the sounds produced by heart and lungs. FIG. 5 shows display 300 showing breath sounds 302 and lung sounds 304, in accordance with one exemplary embodiment of the present invention. FIG. 6 shows display 400 showing speaker 402, and ECG heart signal 404 and corresponding ECG waveform (Electrocardiographic Tracing) 406, in accordance with one exemplary embodiment of the present invention.

In addition, medical instrument 200 encompasses memory 240 for storing the data recorded by diaphragm 204 and the data processed by microcontroller 210. Microcontroller 210 controls real-time clock 242 of medical instrument 200. Medical instrument 200 receives power from a rechargeable battery 244 via AC/DC adapter 246.

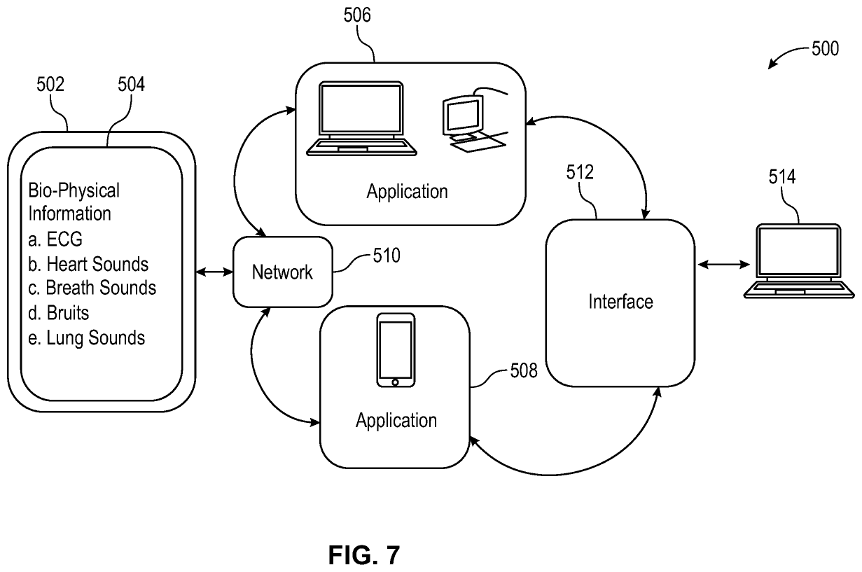

FIG. 7 shows a network architecture 500 of medical instrument 502 communicating with server 514 operated/managed by a telemedicine provider/hospital/government/third party telehealth service provider. Here, medical instrument 502 includes data or bio-physical information 504 of a user (not shown) such as ECG, heart sounds, breath sounds, bruits, lung sounds, etc. Medical instrument 502 communicatively connects to user devices 506, 508 such as mobile devices, laptops, etc. via network 510. In one example, each of user devices 506, 508 includes a software application specifically configured to interact with medical instrument 502 and server 514. In one example, user devices 506, 508 utilize an interface 512 specifically interact with server 514 via network 510. Network 510 includes, but not limited to, Bluetooth, internet, Wi-Fi, Li-Fi, wired network.

Medical instrument 502 connects to server 514 and helps to analyse the heart rate, rhythm (both regular and irregular) and changes, and connects the user to telehealth service provider to obtain current therapies for heart diseases and encourages lifestyle changes as well as supplements and medications for healthcare management.

The presently disclosed medical instrument integrates with other medical devices and help to provide telemedicine to patients. The medical instrument captures and streams/transmits the patient's heartbeat, breathing, blood flow, digestion, or any other biological process to medical practitioners. Further, the medical instrument presents a dashboard with options to integrate and match workflow of an in-person visit in a virtual/online world. For instance, an on-site medical practitioner captures patient's heartbeat, breathing, blood flow, digestion, or any other biological process, prepares intake material or information and submits to the remote service provider/healthcare professional. The remote service provider/healthcare professional securely accesses the information, and reviews the information before consulting the patient. This helps the remote service provider/healthcare professional to have full information about the patient. The medical instrument helps to provide the required consultation to the patients at a relatively low cost.

In addition, the presently disclosed medical instrument provides several advantages over the prior art. The medical instrument gathers data for clinical application and assessment of the patient's condition. The physician or healthcare provider uses that information for clinical purposes. The medical instrument acquires heart and other regional sounds and transposes them on the LCD screen with ECG tracings, physiologic sounds, blood pressure, and oxygen saturation. The presently disclosed medical instrument sensitively picks up normal heart sounds S1, S2, and abnormal heart sounds, S3, S4, and murmurs, heaves, and thrills. Further, the presently disclosed medical instrument displays a diagram showing the patient examiner the locations of the stethoscope placement on the chest and body to maximize the likelihood of picking up both normal and abnormal heart sounds. The medical instrument shows additional diagram(s) to identify the placement of the device for the identification of bruits (carotid, aorta, ophthalmic and renal artery). The presently disclosed medical instrument gathers as much biological data as possible to aid in forming the diagnosis of the remote patient from the diagnostician.

The presently disclosed medical instrument acts as a sensitive mobile stethoscope dedicated to simultaneous ECG tracing, heart, lung, and abdominal sounds, blood pressure, pulse, oxygen saturation, and respiratory rate evaluation. The medical instrument enhances evaluations of the patients located in environments remote from their healthcare provider and transmit relevant high-quality heart, lung, and abdominal sound data and ECG tracings to their care provider through the internet to deliver biological and clinical data to a healthcare practitioner remote from the patient. The medical instrument assists the physician or healthcare practitioner in making diagnostic suggestions based on the clinical data.

The medical instrument has applications in academic/education setting, addiction treatment, community health centers, correctional facilities, hospitals/health systems, physician services, retail/pharmacy, school-based clinics, skilled nursing facilities, urgent care, etc.

The presently disclosed medical instrument operates with third party applications/software such as On-call Health,™ Nextgen Virtual Visits™, Mend™, WebPT™, Simplepractice™, Modernizing Medicine™, Therapy Notes™, Updox™, Vivadox™, Kareo Billing™, Zoom™, Webex™ and any other service providers for telehealth or telemedicine applications.

A person skilled in the art appreciates that the medical instrument may come in a variety of shapes and sizes depending on the need and comfort of the user. Further, many changes in the design and placement of components may take place without deviating from the scope of the presently disclosed medical instrument.

In the above description, numerous specific details are set forth such as examples of some embodiments, specific components, devices, methods, in order to provide a thorough understanding of embodiments of the present disclosure. It will be apparent to a person of ordinary skill in the art that these specific details need not be employed, and should not be construed to limit the scope of the disclosure.

In the development of any actual implementation, numerous implementation-specific decisions must be made to achieve the developer's specific goals, such as compliance with system-related and business-related constraints. Such a development effort might be complex and time-consuming, but may nevertheless be a routine undertaking of design, fabrication, and manufacture for those of ordinary skill. Hence as various changes could be made in the above constructions without departing from the scope of the invention, it is intended that all matter contained in the above description or shown in the accompanying drawings shall be interpreted as illustrative and not in a limiting sense.

The foregoing description of embodiments is provided to enable any person skilled in the art to make and use the invention. Various modifications to these embodiments will be readily apparent to those skilled in the art, and the novel principles and invention disclosed herein may be applied to other embodiments without the use of the innovative faculty. It is contemplated that additional embodiments are within the spirit and true scope of the disclosed invention.

What is claimed is:

1. A medical instrument for assessing vital signs of a living subject in a telemedicine application, the medical instrument comprising:
an elongated member having an Electrocardiography (ECG) sensor;
an extending member, wherein the extending member extends from said elongated member; and
a central member connecting said elongated member, wherein said central member comprises a diaphragm at one end and a bell at another end,
wherein said ECG sensor records electrical heart tracings (ECG tracings) of said living subject, wherein said diaphragm amplifies and produces audio signals associated with one of heartbeat, breathing, blood flow, and digestion of said living subject, and wherein said medical instrument super-imposes a digital visual representation of the audio signals and the ECG tracings into an audio file and transmits to a server for analysing the audio file.

2. The medical instrument of claim 1, wherein said diaphragm detects the sounds produced by heart and lungs of said living subject.

3. The medical instrument of claim 2, further comprises a two channel microphone preamp, wherein said two channel microphone preamp amplifies the sounds using voltage reference and ensures the audio signal is not clipped or distorted and supplies the audio signals to an audio codec for encoding the audio file.

4. The medical instrument of claim 1, further comprises a wireless communication module to transmit the audio file to said server.

5. The medical instrument of claim 1, further comprises a display screen, wherein said display screen displays sounds produced by the heart and lungs.

6. The medical instrument of claim 1, further comprises a speaker, wherein said speaker announces the sounds recorded by said medical instrument.

7. The medical instrument of claim 1, wherein said diaphragm amplifies the audio signals up to forty-five times to produce the audio signals associated with one of the heartbeat, breathing, blood flow, and digestion of said living subject.

8. The medical instrument of claim 1, wherein said server analyses the heart rate, rhythm changes, and recommends the living subject to current therapies for heart diseases and medications for healthcare management.

9. The medical instrument of claim 1, comprises a battery to power electrical components in said medical instrument.

10. A medical instrument facilitating assessment of vital signs of a living subject remotely and in a telemedicine application, the medical instrument comprising:
an elongated member having an Electrocardiography (ECG) sensor;
an extending member, wherein the extending member extends from said elongated member; and
a central member connecting said elongated member, wherein said central member comprises a diaphragm at one end and a bell at another end,
wherein said ECG sensor records electrical heart tracings (ECG tracings) of said living subject, wherein said diaphragm amplifies and produces audio signals associated with one of heartbeat, breathing, blood flow, and digestion of said living subject, wherein said medical instrument super-imposes a digital visual representation of the audio signals and the ECG tracings into an audio file and transmits to a server, and wherein said server analyses the audio file to determine the heart rate, rhythm changes, and provides recommendations the said living subject to current therapies for heart diseases and medications for healthcare management.

11. The medical instrument of claim 10, wherein said diaphragm detects the sounds produced by heart and lungs of said living subject.

12. The medical instrument of claim 11, further comprises a two channel microphone preamp, wherein said two channel microphone preamp amplifies the sounds using voltage reference and ensures the audio signal is not clipped or distorted and supplies the audio signals to an audio codec for encoding the audio file.

13. The medical instrument of claim 10, further comprises a wireless communication module to transmit the audio file to said server.

14. The medical instrument of claim 10, further comprises a display screen, wherein said display screen displays sounds produced by the heart and lungs.

15. The medical instrument of claim 10, further comprises a speaker, wherein said speaker announces the sounds recorded by said medical instrument.

16. The medical instrument of claim 10, wherein said diaphragm amplifies the audio signals up to forty-five times to produce the audio signals associated with one of the heartbeat, breathing, blood flow, and digestion of said living subject.

17. A method of assessing vital signs of a living subject, the method comprising the steps of:
providing an Electrocardiography (ECG) sensor at an elongated member;
providing a central member connecting said elongated member, said central member comprising a diaphragm at one end and a bell at another end;
recording electrical heart tracings (ECG tracings) using said ECG sensor;
amplifying and producing audio signals associated with one of heartbeat, breathing, blood flow, and digestion using said diaphragm of said living subject;
super-imposing a digital visual representation of the audio signals and the ECG tracings into an audio file; and
transmitting the audio file to a server for analysing the audio file.

18. The method of claim 17, further comprising:
providing a two channel microphone preamp;
amplifying the sounds using voltage reference and ensuring the audio signal is not clipped or distorted; and
encoding the audio file.

19. The method of claim 17, wherein the step of analysing the audio signals and ECG tracings comprises:
analysing the heart rate, and rhythm changes; and
providing recommendation to said living subject to current therapies for heart diseases and medications for healthcare management.

20. The method of claim 17, wherein the step of amplifying and producing audio signals comprising: amplifying the audio signals up to forty-five times for producing the audio signals associated with one of heartbeat, breathing, blood flow, and digestion of said living subject.

21. The method of claim 17, wherein the step of blood pressure monitoring, the device receives wireless signals from a digital sphygmomanometer; the numerical systolic and diastolic pressures are stored and superimposed on the displays of heart, lung, and abdominal sounds.

22. The method of claim 17, wherein the step of oxygen saturation monitoring, the device receives wireless signals from a pulse oximeter; the numerical saturation percentage is stored and superimposed on the displays of heart, lung, and abdominal sounds.
